(19) United States
(12) Patent Application Publication
(10) Pub. No.: US 2026/0335284 A1
(43) Pub. Date: Oct. 29, 2026

(54) STABLE MITOMYCIN CONCENTRATES

(71) Applicants: Harshal Prabhakar Bhagwatwar, Pune (IN); Manoj Nerurkar, Bangalore (IN); Haripriya Potti, Hyderabad (IN); Suhas S Lavhekar, Nanded (IN)

(72) Inventors: Harshal Prabhakar Bhagwatwar, Pune (IN); Manoj Nerurkar, Bangalore (IN); Haripriya Potti, Hyderabad (IN); Suhas S Lavhekar, Nanded (IN)

(73) Assignee: STEPS BIOSCIENCES, INC., Newark, NJ (US)

(21) Appl. No.: 19/119,566

(22) PCT Filed: Oct. 3, 2023
(86) PCT No.: PCT/IB2023/059902

§ 371 Date: Apr. 9, 2025

(30) Foreign Application Priority Data
Oct. 12, 2022 (IN) 202241058449

(51) Int. Cl. A61K 31/407; A61K 9/08; A61K 47/10; A61K 47/18; A61K 47/20; A61K 47/22

(52) U.S. Cl. CPC A61K 31/407; A61K 9/08; A61K 47/10; A61K 47/18; A61K 47/20; A61K 47/22

(57) ABSTRACT

The present disclosure provides a stable, non-aqueous liquid composition of mitomycin C. In some embodiments, the liquid composition is in the form of non-aqueous liquid concentrates with high concentrations of mitomycin C. Methods of preparing the liquid compositions and methods of using the concentrates in clinical therapy are also described.

### CROSS-REFERENCE TO RELATED APPLICATIONS

The present application is a national stage application of PCT/IB2023/059902, filed Oct. 3, 2023, which claims priority to Indian Provisional application 202241058449 filed on Oct. 12, 2022, which are incorporated herewith by reference.

### FIELD OF THE INVENTION

The disclosure relates generally to highly concentrated, stable, non-aqueous formulations of Mitomycin C (MMC) for the treatment of cancer and other disease conditions. The formulations of the present invention have exceptional stability, and may be injected directly or diluted with sterile water for injection or other suitable media acceptable for parenteral administration. Also provided are methods of manufacture and use of the formulations in clinical therapy.

### BACKGROUND OF THE INVENTION

MMC is an antineoplastic antibiotic isolated from Streptomyces Caespitosus. It is used in the treatment of various types of cancer including gastric, pancreatic, breast, head and neck, biliary, lung, bladder, and cervical cancers. It is available as a lyophilized powder comprising MMC and mannitol (5, 20 and 40 mg vials; Accord), which is to be reconstituted with water for injection to achieve an MMC concentration of 0.5 mg/mL. This solution is approved to be administered intravenously (IV) via an in-dwelling catheter directly, or for slow continuous IV infusion after suitable dilution. This solution is stable for 14 days under refrigeration, or 7 days at room temperature, and must be discarded if unused. More prevalent in clinical practice is further dilution of the reconstituted solution to a concentration of 20 to 40 micrograms of MMC per mL in a variety of administration vehicles such as normal saline, Ringer's lactate, etc., for slow IV infusion. This solution has a stability of less than 24 hours once prepared.

Further, the dosage of MMC is based on body surface area (BSA), with a maximum dosage of 20 mg/m² BSA. Dosage modification is required and needs to be tailored individually based on the toxicity experienced. There is thus wastage of unused reconstituted product.

More recently, a modified release product of MMC for local pyelocalyceal administration was approved in the United States for the treatment of bladder cancer. The product commercially available under the brand name JELMYTO™ (UroGen Pharma, Inc.) is based on the formation of a slow-release gel inside the bladder from a liquid thermo-reversible hydrogel composition. The product is provided as two vials of 40 mg lyophilized MMC each, along with 80 mg of mannitol, and a third vial containing 20 mL of the liquid hydrogel composition. The MMC vials are combined with the hydrogel through a series of extremely complex mixing steps conducted at a low temperature of 2-8 °C, resulting in unsatisfactory mixing and unsatisfactory administration of the product. The product should be used immediately after reconstitution and should be discarded if unused due to rapid degradation of the MMC in solution. Thus, flexibility in the speed and timing of reconstitution would greatly help in this treatment modality. A concentrated composition of MMC allowing mixing of 80 mg of MMC rapidly with the hydrogel composition would be a tremendous value addition to this therapy.

EP 0415430A1 (the '430 application) describes non-aqueous compositions comprising MMC. The '430 application describes the need for a stable solution formulation of MMC. Solubilities in a variety of non-aqueous solvents are provided, as well as stability in different solvents such as polyethylene glycol (PEG), propylene glycol (PG), dimethylacetamide (DMA), and other solvents. MMC was very unstable in water and also in PEG. Solutions at 5 mg/mL were studied and were found to be most stable in a composition comprising PG with a finite percentage of water (5%). Saturation solubilities in the different solvents are not provided, specifically DMA, n-methyl pyrrolidone (NMP), dimethyl sulfoxide (DMSO), or mixtures with other solvents such as PEG, PG and others. There is no description of highly concentrated solutions of MMC in any solvent, or combinations or the need or utility of such solutions in pharmaceutical and clinical practice.

There is thus a need for highly concentrated, non-aqueous solutions of MMC which are physically and chemically stable and allow for rapid dilution/mixing without going through complex, multi-step reconstitution processes. Such compositions and processes to manufacture such compositions are described herein.

### SUMMARY OF THE INVENTION

A liquid composition for the delivery of MMC is described. More specifically, the present disclosure describes non-aqueous solutions of MMC which are highly concentrated and resistant to physical or chemical instability. Processes to manufacture such compositions and their use in clinical practice are also described.

In certain embodiments, such compositions are highly concentrated, with MMC concentrations in excess of the 5 mg/mL concentration reported in the literature; concentrations can be 5-10 mg/mL or higher, and could be as high as the saturation solubility of the MMC in the respective solvent or solvent mixtures. Concentrations of close to 300 mg/ml are achievable with solvents such as DMA, which is approved for human use in parenteral formulations. In more specific embodiments of the invention, MMC concentrations of 40-80 mg/mL are envisaged. Further embodiments envisage concentrations of 10 mg/mL; 20 mg/mL; 40 mg/mL; 60 mg/mL; 80 mg/mL; 100 mg/mL, 120 mg/mL, 140 mg/mL; 160 mg/mL; 200 mg/mL; and 240 mg/mL to allow for rapid dilution. Thus, the higher the concentration, the smaller the volume of the injection. Such compositions of high MMC concentration can be achieved through novel combinations of non-aqueous solvents, with or without the presence of water. In certain aspects of this embodiment, the non-aqueous solvents are water-miscible. In other aspects, the non-aqueous solvents are water immiscible. In further aspects of this embodiment, the compositions use mixtures of water-miscible and immiscible organic solvents. The presence of low amounts of water is well within the scope of the invention, although the presence of water is known to be detrimental to the stability of MMC.

Certain embodiments describe compositions with highly reduced levels of water, such as for example, less than 5% w/w, preferably less than 1% w/w, and more preferably less than 0.5% w/w water in the composition. Compositions free from water are covered within the scope of this invention. In these embodiments with an extremely low moisture content, MMC concentrates of exceptional physical and chemical stability are produced. Such compositions allow for reduced reconstitution times in infusion clinics and also allow for reduced wastage of the composition.

In other aspects of this embodiment are envisaged MMC concentrates as described above with added preservatives to allow for multiple doses of MMC to be dispensed from the same container; thereby improving manufacturing, transportation, storage efficiencies, and clinical efficiencies.

In further embodiments, the MMC is chemically stable for extended periods of time upon storage. Further, such compositions are also physically stable with no incidence of crystallization and other signs of physical instability.

Certain embodiments describe compositions which are readily miscible with delivery fluids such as water for injection, and normal saline among others. This allows for the compositions to be injected directly into infusion bottles/bags just before administration, allowing for reduced reconstitution time. In further embodiments, multiple dose containers are provided which allow for the tailoring of the amount of the composition which is required to be diluted, where the amount is suited to the patient being treated. This allows for reduced wastage of the composition.

The MMC concentrates of the invention are readily miscible with the hydrogel component of JEMLYTO™ described above in all proportions. The reconstitution process as described in the JELMYTO™ label can thus be simplified using the concentrates of the invention. Processes to manufacture such compositions and their use in clinical practice are also described.

### DETAILED DESCRIPTION OF THE INVENTION

The present inventors have surprisingly found that pharmaceutical compositions comprising highly concentrated solutions of MMC in non-aqueous organic solvents can be prepared by using a judicious selection of organic solvents. Such formulations demonstrate exceptional stability. Another key aspect of the invention is that compositions with exceptional stability can be achieved through a reduction in the water content of the composition.

The term “highly concentrated” as defined in the invention is intended to encompass solutions of MMC with concentrations significantly in excess of the 5 mg/mL which is described in the prior art. Such solutions would thus have MMC concentrations in excess of for example 5 mg/mL, or 10 mg/mL, or 20 mg/mL, or 40 mg/mL, up to 250 mg/mL, up to the saturation solubility of MMC in the solvent, depending on the non-aqueous solvents used or combination of non-aqueous solvents used.

Non-aqueous organic solvents of the present invention are of two types: water-miscible and water-immiscible organic solvents. Water-miscible organic solvents could be any solvents which are readily miscible with water in all proportions. Such solvents could include, for example DMA, dimethyl formamide, DMSO, NMP, 2-pyrrolidone, glycerol, PEG, PG, ethanol, and the like, without any limitations. Other organic solvents which are miscible with water in all proportions are all included within the scope of the invention without limitation.

Water-immiscible organic solvents could be organic solvents which are partially miscible to completely immiscible with water. Such solvents could include for example triacetin, benzyl benzoate, triethyl citrate, propylene carbonate, benzyl alcohol, and the like, without limitations.

The non-aqueous organic solvents of the invention are proven to be safe for human parenteral administration. Any organic solvent acceptable for human parenteral administration and mixtures of such solvents are within the scope of this invention. Thus, the composition could comprise for example a solution of MMC in a solvent such as PEG, or PG which have a lower solubility for MMC, to achieve compositions with lower concentrations of MMC; up to for example 10-20 mg/mL. For compositions with high concentrations, such as for example 30-300 mg/mL of MMC, other solvents such as DMA, NMP, DMSO, and the like, can be used. For intermediate concentrations of MMC, a judicious combination of solvents can be used to achieve the desired concentrations. In some embodiments, the MMC may be partially solubilized and partially in suspension. The proportion of the MMC in solution or in suspension can be decided based on the combination of solvents used and the defined final use of the composition. Use of fine suspensions would allow for a highly concentrated composition of MMC, whereby very low amounts of the organic solvents would be used in clinical practice. This is one of the advantages of the invention: the overall reduction in the amounts of organic solvents used.

In certain embodiments, mixtures of non-aqueous water-miscible and water-immiscible organic solvents are envisaged. For example, MMC solutions in a water-miscible solvent could be mixed with a water-immiscible solvent to enhance stability. Thus, in this embodiment, a MMC concentrate in a water-miscible organic solvent could be mixed with a water-immiscible organic solvent in different ratios as long as the MMC stays in solution. The ratio of the solvents could range from 0 to 100% w/w to 100 to 0% w/w.

The MMC concentrates of the invention could also contain other additives such as preservatives, antioxidants, pH adjusting or buffering agents, and the like. The compositions could be filled into suitable packaging materials such as ampoules, single or multiple dose vials, prefilled syringes, and the like, without limitation, provided the basic requirements of the invention are met. MMC concentrates filled into prefilled syringes are a special embodiment of the invention.

An important embodiment of the invention is the preparation of MMC concentrates of exceptional stability. Such concentrates are prepared through the reduction of the moisture content of the compositions. Compositions with less than 1% w/w moisture provide exceptional chemical stability. In further aspects of this embodiment, moisture contents as low as 0.8%, or 0.6%, or 0.4%, or 0.2%, or lower are beneficial for the chemical stability of MMC. The lower the moisture content, the better the stability.

A preferred composition of the invention is an MMC concentrate in neat DMA with less than 0.1% w/w moisture. This concentrate is exceptionally stable at refrigeration temperatures and room temperature.

The reduction in the moisture content could be achieved through a combination of methods.
1. Through the use of MMC and all solvents, and packaging materials of a very low moisture content;
2. Drying the MMC and other materials as part of the process to reduce the moisture content;
3. Reducing the moisture content of the solvents by passing them through a bed of activated molecular sieves;
4. Preparing the MMC concentrate and then passing the concentrate through a bed of the above-mentioned molecular sieves until a moisture content of less than about 1% w/w is achieved.

The MMC could be dried by, for example, recrystalizing the MMC in the last stage of the synthetic process from a dry organic solvent in an atmosphere with low relative humidity. Another method could include, for example, subjecting the API to an environment of low humidity in a drying chamber containing a suitable desiccant. Processes to reduce the moisture content of drug substances are well known in the art of manufacturing and packaging of active ingredients and are included herein in their entirety without limitation. MMC with a low moisture content is available from commercial MMC manufacturers. A dry API is then stored in suitable containers in a dry environment. Secondary packaging such as aluminum double laminated bags with desiccant pouches could be used to store the canisters containing the dry mitomycin. Suitable precautions should be taken while opening the container for formulation processing, such as the control of the relative humidity in the area to levels below about 40% or lower, and the like. Procedures to handle moisture sensitive materials are well known in the art and are all incorporated herein by reference without limitation.

Alternatively, the MMC can be further dried by incorporating the MMC in the solvents of the invention, and then further passing the solution through a bed of moisture adsorbents, such as for example, molecular sieves, to obtain a solution with low moisture contents.

Thus, the process for the manufacture of such MMC concentrates involves the steps of:
1. Providing a solvent or mixture of solvents in which the MMC is soluble;
2. Dissolving the MMC in the solvent system;
3. Passing the solution through a bed of molecular sieves to reduce the moisture content to below 1% w/w;
4. Filtering the solution through a suitable sterilizing filter;
5. Filling the concentrate in the appropriate packaging material such as vials, pre-filled syringes, etc., as required; and
6. Maintaining a low moisture atmosphere during compounding and filling.

Preparing compositions using low moisture DMA and other solvents and MMC with a low moisture content allows for the preparation of the concentrate without passing through the molecular sieves. Alternatively, it is envisaged that the solvent or solvent mixtures are passed through the molecular sieves to reduce the moisture content, and then the MMC is dissolved therein.

The term “molecular sieves” as used in this description is intended to include multiple forms of desiccants which, upon coming in contact with the liquids of the invention, have the capability to extract moisture. More generally, molecular sieves are crystalline metal silicates. These uniformly sized materials have very small holes of uniform size distributed throughout their matrix. Sieves of various pore sizes are available in the market commercially. The molecular sieves are themselves available in different particle sizes (4-12 mesh and the like) and shapes. Attention is directed to the Sigma-Aldrich catalog section of molecular sieves for example for a more detailed description of the different types and categories of molecular sieves available commercially for different applications. This is a subset of such molecular sieves and any other form of molecular sieve is included within the scope of this invention without limitation. As long as the sieve utilized is capable of reducing the moisture content from the MMC concentrates or the solvents of the invention, they are included herein in entirety. The sieves themselves can then be regenerated by heating in order to remove the entrapped moisture from within the pores.

For utilizing the molecular sieves for reducing the moisture, the molecular sieves can be added directly to the MMC concentrate followed by mixing until the moisture content is reduced. Samples of the concentrate can be removed periodically to test for moisture content. The beads are subsequently removed, and the concentrate is then subjected to normal pharmaceutical processing. Alternatively, the molecular sieves can be packed into cylinders or tubes to create columns or beds packed with the sieves and the solvents or the MMC concentrates can be passed through the beds until the moisture content is reduced as described above. The type and amount of beads required for the reduction of moisture will depend on the level of moisture in the solvent or concentrate, the type of sieve selected and its moisture adsorbing capacity, and are all within the scope of routine experimentation. In a packed bed, for example, as described above the liquid could be recirculated through the bed multiple times using for example a pumping means till the moisture content is reduced to the desired level. Whatever the means used the final moisture content should be as described above. It is preferable to use a molecular sieve which is acceptable for pharmaceutical use.

Solvents of the invention are also available commercially with a low moisture content. Use of such solvents directly, with or without the use of further drying, is also within the scope of the invention.

The MMC concentrates could be aseptically filtered through 0.22-micron filters of suitable grade material, such as PTFE and the like, which are compatible with the solvents of the invention. Any suitable method of sterilization of the composition is within the scope of the invention. Any process of reducing the moisture content to less than about 1% is within the scope of the invention as long as the final composition has a moisture content of less than 1% w/w.

The MMC concentrates of the invention can be administered by a slow intravenous push. For direct intravenous administration, a higher concentration of MMC may be preferred to reduce the amount of solvent used, and to minimize pain and irritation at the injection site. For intravenous infusion, the required volume of the MMC concentrate can be added to the relevant infusion fluid to get instantaneous mixing of the solutions and formation of the infusion.

For use with JELMYTO™ for example, the reversible thermal hydrogel can be mixed with the desired amount of the MMC concentrate. The MMC concentrate mixes rapidly with the hydrogel. Less than about 5 minutes are required to get a clear solution when compared with the tedious procedure referenced in the JELMYTO™ label.

The following examples further describe specific embodiments of the invention in greater detail.

### EXAMPLES

### Example 1: Solubility of MMC in Different Solvents

MMC saturation solubility at 25 °C was evaluated in different solvents. The solubility was determined by adding weighed aliquots of MMC to 1 g of individual solvents with vortex mixing and sonication until MMC undissolved particles were observed. The resulting solutions were subjected to filtration through 0.22-micron PTFE filters and subsequently analyzed by a specific, stability-indicating HPLC method. Table 1 describes the saturation solubility of MMC in neat individual water-miscible and water-immiscible organic solvents.

TABLE 1
Solubility of MMC in individual solvents

| No. | Solvent | MMC solubility in mg/g |
|---|---|---|
| 1 | Water for injection | 2.1 |
| 2 | Propylene glycol | 12.5 |
| 3 | Polyethylene Glycol 400 | 27.0 |
| 4 | Dimethyl Sulfoxide | 213.6 |
| 5 | N-methyl Pyrrolidone | 188.9 |
| 6 | Dimethyl acetamide | 298.1 |
| 7 | Benzyl alcohol | 16.6 |

MMC is very poorly soluble in water, but demonstrates exceptionally high solubility in DMA, DMSO, and NMP. PEG and PG demonstrate lower MMC solubility, while benzyl alcohol, a poorly miscible to water-immiscible organic solvent, also demonstrates low MMC solubility.

### Example 2: Preparation of MMC Concentrates in Mixtures of Water-Miscible Organic Solvents

Solutions of MMC in mixtures of organic solvents were prepared as per Example 1. Table 2 describes the results of MMC solubility in mixtures of water-miscible organic solvents.

TABLE 2
Solubility of MMC in mixtures of water-miscible solvents

| No. | Solvent | MMC solubility, mg/g |
|---|---|---|
| 1. | PG and PEG-400 (80:20) | 15.9 |
| 2. | PG and PEG-400 (50:50) | 24.1 |
| 3. | PG and BA (90:10) | 13.2 |
| 4. | PG and DMSO (80:20) | 19.8 |
| 5. | PG and DMA (80:20) | 14.7 |
| 6. | PG and NMP (80:20) | 25.6 |
| 7. | PEG-400 and DMSO | 99.4 |
| | (80:20) | |
| 8. | PEG-400 and DMA (80:20) | 74.1 |
| 9. | PEG-400 and NMP (80:20) | 76.4 |
| 10. | PEG-400 and DMSO | 102.4 |
| | (70:30) | |
| 11. | PEG-400 and DMA (70:30) | 105.1 |
| 12. | PEG-400 and NMP (70:30) | 84.8 |

The data clearly indicates the formation of MMC concentrates with concentrations above the 5-10 mg/mL reported in the literature. The use of PEG 400 in combination with strong solvents such as DMSO, DMA, or NMP resulted in concentrates with much higher MMC concentrations than with PG combinations.

The data clearly indicates that pharmaceutically relevant concentrations of 20, 40 and 80 mg/mL can be readily achieved through a judicious use of combinations of PEG 400 and one of either DMA or DMSO or NMP. The lyophilized product on the market is available in the form of 5 or 20 or 40 mg vials and doses as high as 80 mg are required per patient.

### Example 3: Stability of MMC Concentrates in Mixtures of Water-Miscible Organic Solvents

An MMC concentrate was prepared at a concentration of 80 mg/mL in a combination of 70:30% w/w of PEG 400:DMA. The concentrate was filled into vials and sealed with rubber plugs and placed in stability testing. Table 3 depicts the performance instability testing.

TABLE 3

| Test | | 2 to 8 °C | | | | | | | 25 °C/60% RH | | | | |
|---|---|---|---|---|---|---|---|---|---|---|---|---|---|
| Parameters | Initial | 7 D | 15 D | 1 M | 2 M | 3 M | 6 M | 7 D | 15 D | 1 M | 2 M | 3 M | 6 M |
| Description | Clear purple colored solution | | | | | | | | | | | | |
| Moisture, % | 0.6 | 0.8 | 0.9 | NA | 1.0 | 1.1 | 0.9 | 1.5 | 1.6 | NA | 1.4 | 1.6 | 1.0 |
| pH | 6.71 | 6.97 | 6.50 | 6.70 | 7.50 | 7.40 | 6.6 | 7.07 | 7.00 | 8.20 | 9.20 | 9.30 | 9.48 |
| Assay, % | 98.6 | 96.0 | 98.2 | 98.2 | 97.9 | 97.7 | 97.7 | 94.5 | 95.1 | 88.1 | 51.5 | 53.2 | 1.0 |
| Total | 0.9 | 0.98 | 1.1 | 1.2 | 1.1 | 1.4 | 1.2 | 1.30 | 3.34 | 12.6 | 50.9 | 54.9 | 98.8 |
| Impurities, | | | | | | | | | | | | | |
| % | | | | | | | | | | | | | |

From the data, it is observed that due to high moisture content and also due to high pH, the rate of degradation is faster in stability testing. In further experiments, the moisture level and pH were controlled to get a stable product.

### Example 4: MMC Concentrates with Reduced Moisture Content

A MMC concentrate was prepared as per Example 3 at a concentration of 80 mg/mL in a mixture of PEG 400 and DMA at a ratio of 70:30. The concentrate thus prepared had a moisture content of 0.76% w/w before treatment. The concentrate was then exposed to molecular sieves (3 A° beads, 4 to 8 mesh; Sigma Aldrich) at a ratio of 0.5:1 (molecular sieves:concentrate) in a sealed container under nitrogen. The mixture was allowed to equilibrate for 7 hours. The concentrate thus obtained was subsequently filtered and filled into vials and stability tested.

TABLE 4

| Test | | 2 to 8 °C | | | | | | | 25 °C/60% RH | | | | | |
|---|---|---|---|---|---|---|---|---|---|---|---|---|---|---|
| Parameters | Initial | 7 D | 15 D | 1 M | 2 M | 3 M | 6 M | 12 M | 7 D | 15 D | 1 M | 2 M | 3 M | 6 M |
| Description | Clear purple colored solution | | | | | | | | | | | | | |
| Moisture | 0.32 | — | — | 0.66 | — | 0.41 | 0.72 | — | — | — | 0.56 | — | 0.42 | 2.7 |
| content, % | | | | | | | | | | | | | | |
| pH | 7.4 | 7.5 | 7.3 | 7.3 | | 6.7 | 7.1 | — | 7.6 | 7.4 | 8.6 | | 9.0 | — |
| Assay, % | 100.0 | 99.9 | 98.9 | 97.5 | 97.8 | 97.6 | 87.5 | 84.6 | 98.3 | 97.7 | 94.0 | 83.3 | 85.2 | 39.2 |
| Total | 0.64 | 0.86 | 0.88 | 0.82 | 0.99 | 1.1 | 0.9 | 4.1 | 0.93 | 1.00 | 4.23 | 12.26 | 10.1 | 49.5 |
| Impurities, % | | | | | | | | | | | | | | |

It is obvious from the above example that a composition with reduced moisture has enhanced stability when compared with a formulation with moisture present at higher levels.

### Example 5: MMC Concentrates with pH Stabilizers

From the examples above, it is apparent that a reduced moisture content has a surprising effect on MMC stability in non-aqueous organic solution concentrates. It is also observed that the pH of the concentrates rises on stability with associated degradation of the MMC.

Several MMC concentrates were thus prepared in different ratios of PEG 400 to DMA as per the procedures outlined above with added buffering for pH control. The placebo compositions with or without alkaline stabilizers were prepared by blending in different predefined ratios of the solvents and passing through molecular sieves to achieve a moisture content below about 0.1% w/w. MMC was then added to the different compositions to achieve a final MMC concentration of 80 mg/mL. The formulations were then filtered and filled into vials and stability tested.

TABLE 5
Mitomycin in PEG 400 and DMA (70:30)

| | | 2 to 8 °C | | | | | 25 °C/60% RH | | | | |
|---|---|---|---|---|---|---|---|---|---|---|---|
| Test Parameters | Initial | 15 D | 1 M | 2 M | 3 M | 6 M | 15 D | 1 M | 2 M | 3 M | 6 M |
| Description | Dark violet to black colored solution | | | | | | | | | | |
| Moisture content, % | 0.03 | — | — | — | 0.13 | 0.16 | — | — | — | 0.15 | 0.21 |
| pH | 7.6 | — | — | — | 7.3 | 7.8 | — | — | — | 7.5 | 9.3 |
| Assay, % | 98.9 | 98.3 | 100.8 | 97.4 | 97.3 | 96.3 | 98.7 | 97.0 | 97.5 | 90.7 | 37.1 |
| Total Impurities, % | 0.39 | 0.47 | 0.42 | 0.45 | 0.45 | 0.46 | 0.57 | 0.51 | 0.79 | 2.82 | 64.58 |

TABLE 6
Mitomycin in PEG 400 and DMA (70:30) + Meglumine

| | | 2 to 8 °C | | | | | 25 °C/60% RH | | | | |
|---|---|---|---|---|---|---|---|---|---|---|---|
| Test Parameters | Initial | 15 D | 1 M | 2 M | 3 M | 6 M | 15 D | 1 M | 2 M | 3 M | 6 M |
| Description | Dark violet to black colored solution | | | | | | | | | | |
| Moisture content, % | 0.05 | — | — | — | 0.12 | 0.13 | — | — | — | 0.17 | 0.21 |
| pH | 7.8 | — | — | — | 7.4 | 8.1 | — | — | — | 7.7 | 9.5 |
| Assay, % | 96.1 | 95.8 | 99.8 | 96.7 | 98.6 | 97.8 | 97.4 | 97.5 | 96.4 | 86.7 | 26.5 |
| Total Impurities, % | 0.35 | 0.46 | 0.43 | 0.45 | 0.47 | 0.43 | 0.55 | 0.51 | 0.82 | 6.50 | 73.35 |

TABLE 7
Mitomycin in plain DMA

| | | 2 to 8 °C | | | | | 25 °C/60% RH | | | | |
|---|---|---|---|---|---|---|---|---|---|---|---|
| Test Parameters | Initial | 15 D | 1 M | 2 M | 3 M | 6 M | 15 D | 1 M | 2 M | 3 M | 6 M |
| Description | Clear purple colored solution | | | | | | | | | | |
| Moisture content, % | 0.06 | — | — | — | 0.17 | 0.18 | — | — | — | 0.21 | 0.23 |
| pH | 7.1 | — | — | — | 6.9 | 8.2 | — | — | — | 7.1 | 8.4 |
| Assay, % | 91.1 | 87.7 | 89.8 | 91.2 | 91.4 | 90.2 | 91.3 | 90.4 | 91.0 | 91.0 | 90.9 |
| Total Impurities, % | 0.22 | 0.35 | 0.35 | 0.40 | 0.38 | 0.36 | 0.45 | 0.43 | 0.44 | 0.44 | 0.44 |

### Example 6: Preparation of MMC Concentrates Using a Mixture of Water-Miscible and Poorly Water-Miscible Organic Solvents

MMC concentrate is prepared at a concentration of 80 mg/mL as per the procedures outlined in the examples above. The solvent mixture used comprises PEG 400, DMA, and benzyl alcohol in a ratio of 70:28:2%. Benzyl alcohol is the poorly water miscible solvent used. A clear dark violet to black colored solution is produced, which is then filled in 10 mL vials. The solution thus prepared serves as a preserved formulation of MMC for multiple dose administration in a hospital setting.

### Example 7: Mixing of the MMC Concentrates with a Reversible Thermal Hydrogel

A hydrogel was prepared as per the JELMYTO™ label composition. Table 8 shows the composition and manufacturing process.

TABLE 8
Composition of Hydrogel

| No. | Ingredients | mg/mL |
|---|---|---|
| 1 | HPMC (EC15) | 2.0 |
| 2 | Poloxamer 407 | 283.5 |
| 3 | PEG 400 | 10.5 |
| 4 | WFI (water for injection) | q.s. to 1 mL |

### Manufacturing Process:

In a suitable glass container, the required quantity of HPMC was dissolved in 50% batch volume of the cooled WFI. Upon complete solubilization, the required quantities of poloxamer 407 and PEG 400 were added and mixed until completely solubilized. The final volume was made up with WFI. The hydrogel bulk solution is filled in 20 mL clear glass vials at a fill volume of 20 mL, flushed with nitrogen, stoppered, and sealed.

Mixing of MMC Concentrate with Hydrogel:

One hydrogel vial and one prefilled syringe containing the MMC concentrate at a concentration of 80 mg/mL were placed in a refrigerator to cool down to 2-8 °C. The syringe and the vial were removed from the refrigerator and the MMC concentrate was injected into the hydrogel by inserting the needle in the rubber plug and inverting the vial while injecting. The gel with the MMC concentrate was gently swirled while maintaining the temperature at 2-8 °C by intermittently placing in an ice-bath for short intervals. The composition formed a clear dark purple solution without any presence of undissolved MMC or any non-uniformity visually. The whole process took less than 5 minutes.

1-15. (canceled)

16. A non-aqueous concentrated solution of mitomycin C, where the solution comprises a non-aqueous solvent or a mixture of non-aqueous solvents and where the concentration of mitomycin C in the solution is 5 mg/ml or higher.

17. The non-aqueous solution of mitomycin C according to claim 16, where the concentration of mitomycin in the solution is from 5 mg/ml to about 10 mg/ml.

18. The non-aqueous solution of mitomycin C according to claim 16, where the concentration of mitomycin in the solution is from about 10 mg/ml to saturation solubility of the mitomycin C in the non-aqueous solvent or the mixture of non-aqueous solvents.

19. The non-aqueous solution of mitomycin C according to claim 16, where the non-aqueous organic solvent is a water-miscible organic solvent, a water-immiscible organic solvent, or a combination thereof.

20. The non-aqueous solution of mitomycin C according to claim 19, where the water-miscible organic solvent is dimethylacetamide (DMA), dimethyl formamide, dimethyl sulfoxide (DMSO), n-methyl pyrrolidone (NMP), 2-pyrrolidone, glycerol, polyethylene glycol (PEG), propylene glycol (PG), ethanol, or a combination thereof.

21. The non-aqueous solution of mitomycin C according to claim 20, where the water-miscible organic solvent is selected from DMA, PEG, and combinations thereof.

22. The non-aqueous solution of mitomycin C according to claim 19, where the water-immiscible organic solvent is triacetin, benzyl benzoate, triethyl citrate, propylene carbonate, benzyl alcohol, or a combination thereof.

23. The non-aqueous solution of mitomycin C according to claim 16, where the solution comprises less than 5% w/w of moisture.

24. The non-aqueous solution of mitomycin C according to claim 16, where the solution comprises less than 1% w/w of moisture.

25. The non-aqueous solution of mitomycin C according to claim 24, where the solution comprises less than 0.5% w/w of moisture.

26. The non-aqueous solution of mitomycin C according to claim 16, where the solution comprises less than 1% of mitomycin C degradation products when stored at 2-8 °C for at least 6 months.

27. The non-aqueous solution of mitomycin C according to claim 16, where the concentration of mitomycin in the solution is from about 10 mg/ml to about 80 mg/ml.

28. The non-aqueous solution of mitomycin C according to claim 16, where the concentration of mitomycin in the solution is from about 10 mg/ml to about 100 mg/ml.

29. The non-aqueous solution of mitomycin C according to claim 16, where the concentration of mitomycin in the solution is from about 10 mg/ml to about 120 mg/ml.

30. The non-aqueous solution of mitomycin C according to claim 16, where the concentration of mitomycin in the solution is from about 10 mg/ml to about 160 mg/ml.

31. The non-aqueous solution of mitomycin C according to claim 16, where the concentration of mitomycin in the solution is from about 10 mg/ml to about 200 mg/ml.

32. The non-aqueous solution of mitomycin C according to claim 16, where the concentration of mitomycin in the solution is from about 10 mg/ml to about 240 mg/ml.

33. The non-aqueous solution of mitomycin C according to claim 16, where the concentration of mitomycin in the solution is from about 10 mg/ml to about 300 mg/ml.

34. A process of preparation of a mitomycin C concentrated solution, comprising
providing a non-aqueous solvent or a mixture of non-aqueous solvents, wherein mitomycin C is soluble in the non-aqueous solvent or mixture of non-aqueous solvents;
adding and dissolving mitomycin C into the non-aqueous solvent or mixture of non-aqueous solvent to form a mitomycin C solution;
optionally drying the mitomycin C solution to reduce the moisture content below 1%; and
filling the mitomycin C solution into a container.

35. The process of claim 34, where the mitomycin C is added in a concentration of 5 mg/ml or higher.
